(19) United States
(12) Patent Application Publication
(10) Pub. No.: US 2025/0001150 A1
(43) Pub. Date: Jan. 2, 2025

(54) APPLICATOR WITH CAMERA AND SENSOR FOR SKIN HEALTH MAPPING

(71) Applicant: L'Oreal, Paris (FR)

(72) Inventors: Gregoire Charraud, Jersey City, NJ (US); Hyosik Dennis Min, Palisades Park, NJ (US); Rafael Feliciano, New Providence, NJ (US)

(73) Assignee: L'Oreal, Paris (FR)

(21) Appl. No.: 18/345,155

(22) Filed: Jun. 30, 2023

(51) Int. Cl. A61M 35/00

(52) U.S. Cl. CPC A61M 35/003; A61M 2205/3303; A61M 2210/04

(57) ABSTRACT

A personal care system comprises an applicator including a body and an applicator surface configured to apply a formula to skin, a position sensor module; a skin condition sensor module, and a processor. The personal care system is configured to obtain, via the position sensor module, position sensor data; calculate, based on the position sensor data, a position of the applicator on the skin; apply a treatment to the skin at the calculated position; determine treatment characteristics at the calculated position; obtain, via the skin condition sensor module, skin condition sensor data; determine, based on the skin condition sensor data, skin condition information at the calculated position; and store the calculated position, treatment characteristics, and skin condition information. The stored information can be used to generate a visualization of a change in skin condition over time, obtain a recommendation from a recommendation engine, or adjust one or more treatments.

### SUMMARY

In one aspect, a personal care system comprises an applicator including a body and an applicator surface coupled to the body, wherein the applicator surface is configured to apply a formula to skin; a position sensor module; a skin condition sensor module; a processor; and memory having stored therein computer-executable instructions configured to cause the personal care system to perform steps comprising obtaining, via the position sensor module, position sensor data; calculating, based on the position sensor data, a position of the applicator on the skin; applying a treatment to the skin at the calculated position; determining treatment characteristics of the treatment applied to the skin at the calculated position; obtaining, via the skin condition sensor module, skin condition sensor data; determining, based on the skin condition sensor data, skin condition information at the calculated position; and storing the calculated position, the treatment characteristics, and the skin condition information.

In some embodiments, the skin condition sensor module includes a skin hydration sensor, a skin elasticity sensor, or a combination thereof. In some embodiments, the position sensor module includes a camera, a proximity sensor, a gyroscope, an accelerometer, or a combination thereof.

In some embodiments, the computer-executable instructions are further configured to cause the personal care system to compare the determined skin condition information with a baseline condition and adjust one or more treatments based on the comparison.

In some embodiments, the personal care system further includes one or more light sources, and the treatment comprises a light treatment or phototherapy treatment.

In another aspect, a method performed by a computer system comprising one or more processors and memory comprises, by the one or more processors, obtaining position sensor data; calculating, based on the position sensor data, a position of an applicator relative to skin of a user; determining treatment characteristics of a treatment applied to the skin at the calculated position; obtaining skin condition sensor data; determining, based on the skin condition sensor data, skin condition information of the skin at the calculated position; and storing the calculated position, the treatment characteristics, and the skin condition information.

In some embodiments, the method further comprises comparing the determined skin condition information with a baseline condition and adjusting one or more treatments based on the comparison. In some embodiments, the method further comprises generating a visualization (such as a 2D or 3D animation) of a change in skin condition over time based on the stored calculated position, the stored treatment characteristics, and the stored skin condition information. In some embodiments, the method further comprises obtaining a recommendation from a recommendation engine or adjusting one or more treatments based on analysis of the stored information.

In some embodiments, the skin condition information comprises an image of the skin at the calculated location, skin elasticity information, skin hydration information, or a combination thereof.

In some embodiments, the treatment characteristics include a timestamp. In some embodiments, the treatment characteristics further include an identifier of a dispensed formula, a quantity of the dispensed formula, or a combination thereof. In some embodiments, the treatment characteristics further include an intensity of treatment, a wavelength of treatment, a duration of treatment, or a combination thereof.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1A is an example applicator, in accordance with the present technology;

FIG. 1B is an example cross-section of the applicator of FIG. 1A, in accordance with the present technology;

FIG. 1C is another example cross-section of the applicator of FIG. 1A, in accordance with the present technology;

FIG. 2A is another example applicator, in accordance with the present technology;

FIG. 2B is an example cross-section of the applicator of FIG. 2A, in accordance with the present technology;

FIG. 2C is another example cross-section of the applicator of FIG. 2A, in accordance with the present technology;

FIGS. 3A and 3B are top views of example applicators, in accordance with the present technology;

FIG. 4 is an example system including an applicator and a base device, in accordance with the present technology;

FIG. 5 is another example system, in accordance with the present technology;

FIG. 6 is an example method of using a system, in accordance with the present technology; and

FIG. 7 is another example method of using a system, in accordance with the present technology.

### DETAILED DESCRIPTION

Described herein is a system for detecting skin conditions and mapping skin health. In some embodiments, a personal care system comprises a treatment and sensing assembly that includes an applicator having a body and an applicator surface coupled to the body, a position sensor module, a skin condition sensor module, and a processor, with the applicator surface being configured to apply a formula to skin. In some embodiments, the applicator is configured to be coupled to a base device in a hand-held form factor. In some embodiments, the personal care system is programmed to perform steps comprising obtaining, via the position sensor module, position sensor data; determining, based on the position sensor data, a position of the applicator on the skin; applying, by the applicator, the formula to the skin at the determined position; determining formula application characteristics of the formula applied to the skin at the determined position; obtaining, via the skin condition sensor module, skin condition sensor data; determining, based on the skin condition sensor data, a skin condition at the determined position; storing the position information, the formula application characteristics, and the skin condition. This stored information can be used by the personal care system or some other computing device or system to, e.g., display a visualization of the treatment and its progressive effects (e.g., in a time lapse video or animation), determine changes in skin condition over time, recommend different products, adapt treatments or routines to changing environmental conditions or skin conditions, or for other purposes.

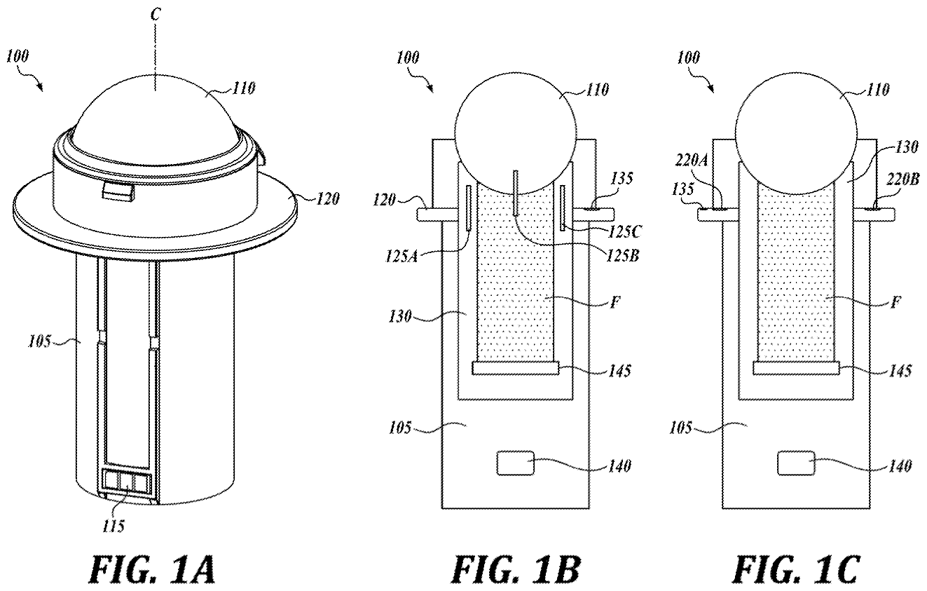

FIGS. 1A-1C illustrate aspects of an example treatment and sensing assembly, in accordance with the present technology. In this example, the treatment and sensing assembly is configured as an applicator 100 for applying formula that also includes sensors that obtain sensor data for calculating position of the applicator on the user's skin, determining skin condition information, or for other purposes. As shown in FIG. 1A, applicator 100 may include a body 105, an applicator surface 110, a tag 115, and a platform 120. Also illustrated is a cross-section line C, along which FIGS. 1B and 1C are taken. In some embodiments, the body 105 is configured to contain additional components and/or circuitry, such as one or more sensor modules, as shown in FIGS. 1B and 1C.

The applicator surface 110 may take any number of forms, including a roller ball configured to distribute and apply a formula located in a reservoir inside the applicator 100 (as shown in FIGS. 1B and 1C). In some embodiments, the roller ball 110 is plastic, but in other embodiments, the roller ball 110 may be glass or metal. In some embodiments, the roller ball is transparent.

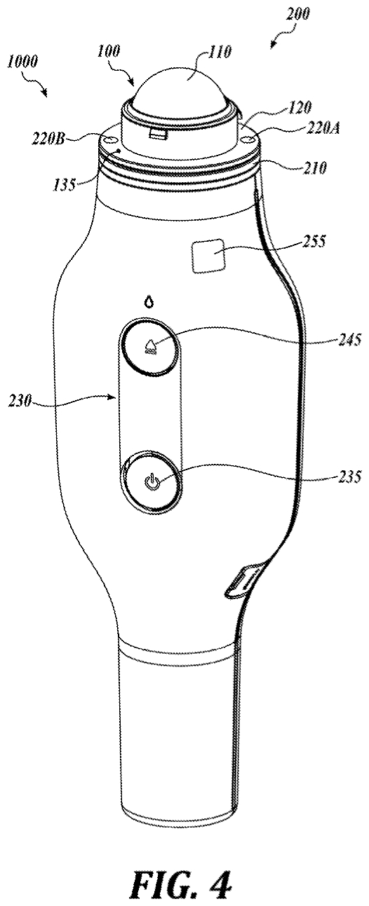

In some embodiments, the applicator 100 further includes a tag 115. In some embodiments, the tag 115 is a quick response (QR) code, radiofrequency identification (RFID) tag, barcode, or the like. In some embodiments, the tag 115 communicates an identity of the applicator 100 or the one or more formulations inside the applicator 100 to a base device, such as shown in FIG. 4. In some embodiments, the tag 115 may be used to identify any number of things about the one or more formulas or applicator 100, including the amount of formula inside the applicator 100, the expiration date of the formula F inside the applicator 100, or when to replace the applicator 100.

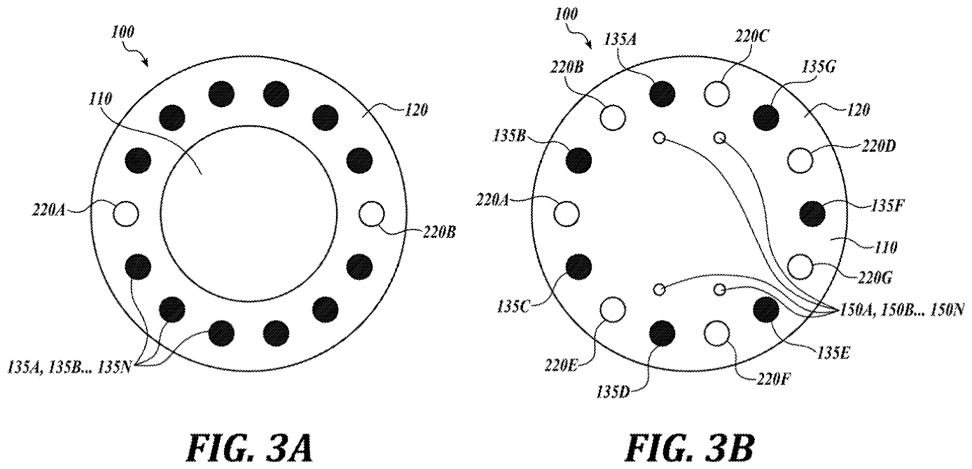

In some embodiments, the applicator 100 is configured to be coupled to a base device, such as the base device shown in FIG. 4. In some embodiments, the applicator 100 includes a platform 120. While the platform 120 is illustrated in FIGS. 1A-1C as including a disk or ring shape, the platform 120 may be configured in other shapes. The platform 120 may include attachment means capable of securing a treatment and sensing assembly (e.g., applicator 100) to a base device 200, such as in a threaded screw-in or screw-on configuration, a snap-in or snap-on configuration, or with a magnet. In some embodiments, the platform 120 is at least partially clear or translucent, which may be useful for, e.g., allowing light from light sources in the top end of the base device to pass through the platform. In some embodiments, the platform 120 may include any number of additional components, such as the components shown in detail in FIGS. 3A and 3B.

In some embodiments, the applicator 100 may be configured to apply one or more specific types of skin treatment, such as formula treatment, light treatment, or radiation treatment. In other embodiments, the applicator 100 may be configured to apply a combination of such treatments. In operation, the applicator 100 may be placed inside a base device (as shown in FIG. 4) and secured to the base device with the platform 120. The roller ball 110 may be rolled over a surface, such as a user's skin, to apply a formula.

In some embodiments, the formula may be a moisturizer, a concealer, a wrinkle or fine line treatment, a toner, an acne treatment, a sunscreen, a foundation, or the like. Multiple formulas may be applied for different purposes, such as for treating different skin conditions or treating a condition with a combination of formulas.

FIGS. 1B and 1C are example cross-sections of the illustrative applicator 100, in accordance with the present technology. In the examples shown in FIGS. 1B and 1C, the applicator 100 includes a body 105, an applicator surface 110, a platform 120, a reservoir 130 configured to hold a formula F, a piston 145, and an applicator processor 240.

In some embodiments, the reservoir 130 is located inside the body 105, and is configured to hold a formula F, such as a skin care formula. In some embodiments, the skin care formula is a moisturizer, a toner, an acne treatment formula, a wrinkle or fine line treatment formula, or a cosmetic. As the applicator surface, illustrated here as a roller ball 110, moves or rolls, formula F from the reservoir 130 is applied to a surface.

In some embodiments, the applicator 100 further includes a piston 145 configured to push the formula F towards the roller ball 110 as the formula is applied. In some embodiments, the piston 145 is controlled by circuitry on a dispensing device or on the applicator 100 itself to push the formula F towards the applicator surface at a defined rate to control the flow rate of the formula F onto the surface of the roller ball 110.

In some embodiments, the applicator 100 includes an applicator processor 140. In some embodiments, applicator processor 140 performs functions relating to application of treatments. In some embodiments, the applicator processor 140 also performs functions relating to obtaining and storing information relating to skin conditions and treatments, and tracking progress of treatments over time.

Regarding functions relating to treatments, in some embodiments the applicator processor 140 is configured to control the piston 145 to push the formula F towards the applicator surface 110 to the formula to the skin, to apply other treatments such as light treatments, or to control other aspects of the applicator 100.

Regarding functions relating to obtaining and storing information relating to skin conditions and treatments, and tracking progress of treatments over time, in some embodiments the applicator processor 140 is further configured to obtain position sensor data from a position sensor module and calculate a position of the applicator on the skin based on the position sensor data. As another example, the applicator processor 140 may further be configured to obtain, via a skin condition sensor module, skin condition sensor data and determine, based on the skin condition sensor data, skin condition information at the calculated position.

FIG. 1B is an example cross-section of the applicator 100 along cross-section line C in FIG. 1A. In the example shown in FIG. 1B, the applicator 100 further includes one or more sensor modules 135, such as a position sensor module and a skin condition sensor module.

In some embodiments, the sensor module 135 includes a skin condition sensor module configured to obtain skin condition sensor data for detecting a condition of a portion of the skin. In some embodiments, the sensor module 135 transmits skin condition data to a processor, such as the applicator processor 140, for analysis. In some embodiments, the skin condition sensor module includes a skin hydration sensor, a skin elasticity sensor, or a combination thereof. A skin elasticity sensor may take the form of a probe device configured to measure mechanical and viscoelastic properties of skin, such as by applying suction to the skin and measuring resistance of the skin to that suction, or through the use of tonometry. A skin hydration sensor may include, e.g., a capacitance sensor or conductance sensor.

In some embodiments, the sensor module 135 includes a position sensor module configured to obtain position sensor data for calculating a position of the applicator 100 on the skin. In some embodiments, the position sensor module includes a two-dimensional (2D) or three-dimensional (3D) camera module, a proximity sensor, a gyroscope, an accelerometer, or a combination thereof. In some embodiments, the camera module includes one or more cameras configured to take one or more 2D or 3D images (i.e., still images or video) of the skin as the user moves the applicator over the skin. In some embodiments, the camera module includes a microscope camera to capture magnified images of the skin area of interest. Capture of images may be aided by illumination provided by one or more light sources, such as light sources positioned on the applicator. Such images may be captured by the camera in, e.g., the visible, infrared, or ultraviolet spectrum, or a combination thereof. Images captured by the camera may be used to calculate the position of the applicator on the skin, such as by comparing captured images with known skin landmarks. Alternatively, position can be calculated based on images captured by one or more cameras positioned at a distance from the applicator, such that the images capture both the applicator itself and the part of the body on which the applicator is located. Other sensors such as accelerometers also may be used to calculate velocity of the applicator across the surface of the skin, orientation of the applicator relative to the skin surface, or other information.

In some embodiments, the applicator 100 is configured to administer radiation treatment via one or more radiating elements 125A, 125B, 125C. While three radiating elements 125A, 125B, 125C are illustrated in FIG. 1B, it should be understood that more or fewer radiating elements 125A, 125B, 125C may be incorporated into applicator 100. In some embodiments, the radiating elements 125A, 125B, 125C include antennas. In some embodiments, the radiating elements 125A, 125B, 125C are located behind and around the applicator surface 110 as shown in FIG. 1B. In some embodiments, the radiating elements 125A, 125B, 125C are configured to produce one or more beams of radiation energy and direct the radiation energy to the skin. In some embodiments, the sensor module 135 includes a sensor module configured to determine an intensity, power, amplitude, or frequency of applied radiation energy. In some embodiments, the radiation treatment comprises phototherapy. In some embodiments, the radiation treatment is ultraviolet (UV) treatment. In some embodiments, the UV treatment is used to treat eczema, psoriasis, jaundice, vitiligo, mycosis fungoides, and the like.

FIG. 1C is another example cross-section of the applicator 100 along cross section line C in FIG. 1A. In some embodiments, the applicator 100 is configured to apply one or more light treatments or to otherwise illuminate the skin with one or more light sources 220A, 220B. While two light sources 220A, 220B are illustrated, it should be understood that more or fewer light sources may be incorporated into the applicator 100. The skin condition data may be sent to the applicator processor 140, which may direct the applicator 100 to apply one or more light treatments or to take some other action, such as to illuminate the skin surface for image capture. In some embodiments, light sources 220A, 220B are configured to administer light treatment, including, but not limited to blue light, yellow light, near infrared light, or a combination thereof.

One skilled in the art should understand that in some embodiments, the applicator 100 may be configured to administer both radiation therapy and light therapy, concurrently or independently. The individual figures should not be interpreted as mutually exclusive. For example, in some embodiments, applicator 100 may include all of the components illustrated in both FIGS. 1B and 1C.

In some embodiments, the applicator processor 140 determines whether to administer one or more treatments, such as applying one or more formulas F from the formula reservoir 130 or applying light treatment, phototherapy treatment, or radiation treatment, or to set or adjust parameters for such treatments. In some embodiments, as treatments are applied, treatment characteristics are collected. In some embodiments, the treatment characteristics include a timestamp of the time the treatment was applied. In some embodiments, the treatment characteristics include environmental data describing the environment in which the treatment was applied, such as ambient temperature, humidity, pollution levels, or the like. In some embodiments, the treatment characteristics include details of the treatment applied, such as an identifier of a dispensed formula, a quantity of the dispensed formula, an identifier of a type of light therapy or phototherapy treatment; an intensity, wavelength, or duration of a type of light therapy or phototherapy treatment, or a combination thereof.

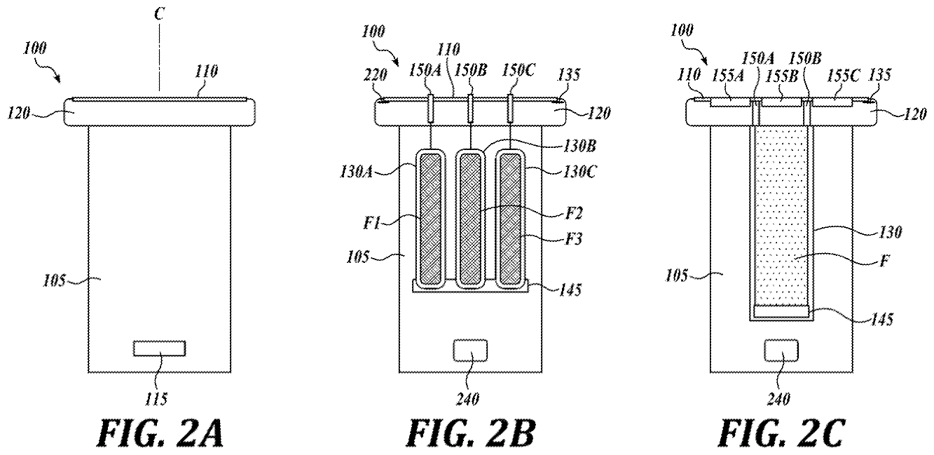

FIG. 2A is another example applicator 100, in accordance with the present technology. In the example shown in FIG. 2A, the applicator includes a flat applicator surface 110. In operation, the applicator surface 110 may be moved across the skin. In some embodiments, the applicator surface 110 is the same size and shape as the platform 120. In some embodiments, the applicator surface 110 is integrated into the platform 120. In some embodiments, the applicator surface 110 includes openings, such to allow formula expelled by one or more nozzles positioned at or below the applicator surface, as shown in FIGS. 2B and 2C. In some embodiments, the applicator surface 110 is at least partially clear or translucent, which may be useful for, e.g., allowing light from light sources in the top end of the base device 200 or on the platform 120 to pass through the applicator surface.

FIGS. 2B and 2C are example cross-sections of an applicator, in accordance with the present technology. In the examples shown in FIGS. 2B and 2C, the applicator 100 includes a body 105, an applicator surface 110, a platform 120, a sensor module 135, one or more formula reservoirs 130 configured to hold a formula F, a piston 145, and an applicator processor 240.

In some embodiments, multiple formula reservoirs (e.g., reservoirs 130A-130C) located inside the body 105 are configured to hold one or more formulas F or formula ingredients. In some embodiments, the one or more formulas F are a skin care formula. In some embodiments, the skin care formula is a moisturizer, a toner, an acne treatment, a wrinkle treatment, fine line treatment, or a cosmetic. As the applicator surface, illustrated here as a flat applicator surface 110, moves across the surface, formula F from the reservoir 130 is applied to the surface through one or more nozzles (e.g., nozzles 150A-150C).

In some embodiments, the applicator 100 further includes a piston 145 configured to push the formula F towards the nozzles 150A, 150B, 150C as the formula F is applied. In some embodiments, the piston 145 is directed by circuitry on a base device or on the applicator itself to push the formula F towards the applicator surface 110.

In some embodiments, the applicator 100 includes an applicator processor 240. In some embodiments, the applicator processor 240 is configured to control components of the applicator to perform one or more functions, as to direct the piston to push the formula F towards the applicator surface 110. In some embodiments, the applicator processor 240 may further be configured to detect a condition of the skin with one or more sensors as described herein, and/or to direct the applicator 100 to apply one or more treatments, as described herein.

In the example shown in FIG. 2B, the applicator 100 includes a plurality of formula reservoirs 130A, 130B, 130C configured to hold formulas F1, F2, F3. In some embodiments, the formula reservoirs 130A, 130B, 130C are fluidly coupled to nozzles 150A, 150B, 150C. In some embodiments, each nozzle is configured to dispense a different formula. In some embodiments, multiple reservoirs are coupled to a single nozzle, which may be useful for mixing formulas or ingredients from separate reservoirs prior to being expelled from the nozzle. In some embodiments, individual reservoirs are coupled to more than one nozzle, which may be useful for distributing the formula across a wider area of the applicator surface.

In the examples shown in FIGS. 2B and 2C, the applicator 100 further includes one or more sensor modules 135, as described herein. In some embodiments, the applicator 100 is configured to administer one or more light treatments with a light source 220. While a single light source 220 is illustrated in FIG. 2B, more or fewer light sources may be incorporated into the applicator 100. In some embodiments, the light treatment is near-infrared (NIR) light treatment, red light treatment, blue light treatment, and/or yellow light treatment.

In some embodiments, the sensor module 135 includes a skin condition sensor module configured to detect a condition of a portion of the skin. In some embodiments, a processor (e.g., applicator processor 240) directs the sensor module 135 to collect skin condition data as the applicator is moved across the skin and receives the collected skin condition data from the sensor module 135. In some embodiments, the applicator processor 240 (or some other internal or external processor or combination of processors) uses the skin condition data collected by the sensor module 135 to determine skin conditions at that location, to determine whether or when to apply treatments to the skin at that location, or to perform some other function.

In the example shown in FIG. 2C, the applicator 100 includes a reservoir 130 configured to hold a formula F, and one or more radiating elements 155A, 155B, 155C on an applicator surface 110 having openings through which nozzles 150A, 150B may expel the formula F.

In some embodiments, the sensor module 135 is configured to obtain skin condition data of at least a portion of the skin and transmit the skin condition data to the applicator processor 240. In some embodiments, the sensor module 135 is configured to obtain intensity, power, amplitude, or frequency data of radiation energy. In some embodiments, the applicator processor 240 is configured to detect a condition of the skin based on sensor data obtained from the sensor module 135 and determine whether to administer one or more treatments, such as applying formula F from the formula reservoir 130, applying radiation treatment, applying light treatment, or combinations thereof.

FIGS. 3A and 3B are top views of example applicators 100, in accordance with the present technology. In some embodiments, sensors of one or more sensor modules, light sources, or both are arranged on the applicator 100 in a ring configuration. In some embodiments, the applicator 100 includes two light sources 220A, 220B located on opposite sides of the ring, as shown in FIG. 3A. In some embodiments, the applicator 100 includes a plurality of sensors 135A, 135B . . . 135N in a ring configuration or other configuration, and the sensors may be associated with one or more sensor modules (e.g., a skin condition sensor module, a position sensor module). In this manner, the applicator 100 can obtain data that allows the applicator (or some other device or system) to determine skin conditions on one or more portions of the skin, as a user moves the applicator 100. In some embodiments, the sensors 135A, 135B . . . 135N can be used to determine skin conditions in a full 360-degree field of view from the applicator surface 110.

In some embodiments, the light sources 220A, 220B, 220C, 220D, 220E, 220F, 220G can be alternated or otherwise interspersed with the one or more sensor modules 135A, 135B, 135C, 135D, 135E, 135F, 135G, as shown in the illustrative configuration of FIG. 3B. In some embodiments, the applicator 100 may both detect a condition of one or more areas of the skin and administer light treatment or phototherapy treatment as the applicator moves across the skin in any direction.

In some embodiments, the user can apply the formula to their skin while light treatment, such as near-infrared (NIR) light treatment, red light treatment, blue light treatment, yellow light treatment, or a combination thereof is applied to further treat the condition. In some embodiments, the user can apply the formula to their skin while radiation treatment is applied, such as X-rays, gamma rays, electrons, protons, neutrons, or a combination thereof to further treat the condition. In some embodiments, the radiation treatment is ultraviolet (UV) light. In some embodiments, the radiation treatment is configured to treat eczema, psoriasis, jaundice, fungal mycoides, and the like.

While FIG. 3A is illustrated as having a roller ball applicator surface 110 and FIG. 3B is illustrated as having a flat applicator surface 110 with a plurality of nozzles 150A, 150B, 150C, 150D, either applicator surface 110 could be incorporated with any arrangement of sensors or light sources disclosed herein, or other arrangements not limited to those shown in FIGS. 3A and 3B.

In some embodiments, one or more of the sensors 135A, 135B, 135C, 135D, 135E, 135F, 135G are configured to measure an impedance, a capacitance, a deflection, a color, a texture, or a combination thereof of the skin. In some embodiments, individual sensors or sensor modules are configured to each measure distinct parameters (capacitance, impedance, deflection, a color, a texture, etc.). In some embodiments, individual sensors or sensor modules are configured to measure a combination of parameters. In some embodiments, duplicate sensors configured to measure the same parameter at different locations on the applicator. In some embodiments, the condition to be detected based on data obtained from such sensors is hyperpigmentation, acne, fine lines, wrinkles, sun damage, hydration, collagen production, elasticity, oiliness, or a combination thereof.

FIG. 4 is an example system 1000 including an applicator 100 and a base device 200, in accordance with the present technology. In the example shown in FIG. 4, the system 100 includes light sources 220A and 220B, actuators 230, and a sensor module 135.

In some embodiments, the base device 200 includes an end 210. The end 210 may be configured to be visible through the platform 120 on the applicator 100. In some embodiments, the end 210 of the base device 200 includes the light sources 220A, 220B, which are configured to emit light for administering light treatment to a surface. The light treatment can be administered while other treatments are carried out, such as while formula is being applied by the applicator 100.

In some embodiments, the light sources 220A, 220B are LEDs. In some embodiments, a first light source 220A is configured to administer light treatment in a first wavelength, and a second light source 220B is configured to administer light treatment in a second wavelength. In some embodiments, the light treatment in the first wavelength and the light treatment in the second wavelength are administered simultaneously.

The actuators 230 may be configured to begin the administration of treatments such as light treatment, application of formula, or other treatment or combinations of treatments. In the example shown in FIG. 4, the one or more actuators 230 include a power button 235 and an application button 245. In some embodiments, the power button 235 is configured to turn the base device on or off, while the application button 245 is configured to administer one or more treatments described herein, including light treatment, radiation treatment, or the application of one or more formulas. In the example shown in FIG. 4, the actuators 230 are illustrated as buttons. In some embodiments, the actuators 230 may be switches, capacitive touch type buttons, dials, or the like.

In some embodiments, the base device 200 further includes a base device processor 255. In some embodiments, the base device processor 255 is configured to direct the applicator 100, the base device 200, or both to perform functions such as collecting sensor data, determining conditions based on collected sensor data, storing collected information, transmitting collected information to other devices, or administering one or more treatments. In some embodiments, the base device 200 also includes a contactless chip reader (not pictured in FIG. 4) to perform functions such as reading information from an RFID tag on the applicator 100.

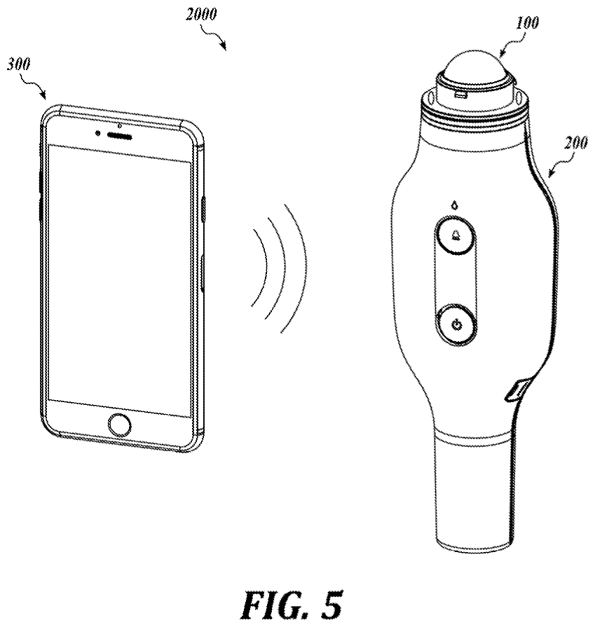

FIG. 5 is another example system 2000, in accordance with the present technology. The system, which implements an applicator 100 and a base device 200 as described herein, further includes a connected communication device 300. Optionally, the system may further include one or more external servers which are implemented as part of a cloud-computing environment. In the example shown in FIG. 5, the connected communication device is a mobile computing device with a touchscreen display, such as a smartphone or tablet computer. In some embodiments, the communication device 300 may be or operate in combination with a desktop computer, a laptop computer, or some other computing device.

In some embodiments, communication device 300 includes one or more cameras and a user interface such as a touchscreen display, which may present to interactive functionality such as depictions of changes in skin conditions over time, tutorial videos or animations, or other elements.

In some embodiments, communication device 300 executes a client application and an image capture/scanning engine configured to capture and process digital images (e.g., color images, infrared images, depth images, etc.) or other data obtain from, e.g., one or more cameras on the applicator 100. In some embodiments, the digital images or scans are processed by communication device 300 and/or transmitted to a remote computer system for processing, e.g., in a 2D or 3D skin surface model engine that maps skin features, skin condition information, or other information in the model. In some embodiments, captured image data is transmitted to a position tracking engine for determining the position of the applicator 100 relative to a body surface. In some embodiments, the position tracking engine maintains awareness of where the target body surface is and where the applicator is in a 3D space.

In some embodiments, digital 3D models described herein are generated based on sensor data obtained from sensor modules 135. In such embodiments, the digital 3D models are generated by the communication device 300 or some other computing device, such as a remote cloud computing system, or a combination thereof. In some embodiments, the digital 3D models include 3D topology and texture information, which can be used for reproducing an accurate representation of a body surface, such as facial structure and skin features.

In some embodiments, communication device 300 prepares information for transmission to, or receives and interprets information from other devices or systems, such as a remote computer system or base device 200. Such information may include captured digital images, scans, or video; device settings; custom care routines specifying, e.g., formula application information or light treatment information; user preferences; usage data, user identifiers, device identifiers, or the like.

In some embodiments, the system 2000 is configured to apply one or more algorithms to a photo or video of a user to detect one or more skin conditions. In some embodiments, the system 2000 uses deep learning algorithms that employ artificial neural networks to identify combinations of image features in input images of skin and classify those input images as including skin conditions such as acne, hyperpigmentation, rosacea, wrinkles, fine lines, wounds, or other conditions. Such algorithms may further take into account other information besides images to identify a condition, such as user questionnaire information. In some embodiments, the deep learning algorithms are trained using a supervised learning approach. In some embodiments, the communication device 300 communicates the detected condition to a user of the system and proposes one or more treatments to be applied by the applicator 100. In some embodiments, the system 2000 may further obtain environmental condition information with one or more environmental sensors or from some other source, such as a server computer. In some embodiments, the environmental conditions may include temperature, humidity, ultraviolet (UV) radiation levels, pollution levels, and the like. In some embodiments, the application can gather environmental data from other sources such as weather services. In some embodiments, the application can further recommend a formula, comprised of one or more skin ingredients, to the user based on the detected skin feature and/or environmental conditions.

In some embodiments, the user can set up a user profile on the application of the communication device 300. In some embodiments, setting up the user profile includes answering a user questionnaire. In some embodiments, the user questionnaire gives the user a series of inputs including past skin treatment, past use of the applicator, desired skin quality, or skin concern. In some embodiments, a client application running on the communication device 300 can solicit feedback from the user regarding their favorite or most effective formulation to help improve the algorithm.

In an embodiment, a care routine engine performs processing of data such as identifying changes in skin condition or generating or adjusting a care routine, such as making adjustments to treatment parameters or recommending new products. The care routine engine may be implemented on the communication device 300 or on some other device or system, such as a server in a cloud computing environment. A care routine may include, for example, programmatic care routine instructions for applying treatments, collecting data (e.g., image data, depth data, usage data, or other data), or performing other functions according to techniques described herein. In an embodiment, the care routine engine generates care routine information based on user information from a user profile data store, a product data store, or some other source or combination of sources. The care routine engine may employ machine learning or artificial intelligence techniques (e.g., template matching, feature extraction and matching, classification, artificial neural networks, deep learning architectures, genetic algorithms, or the like). For example, to identify changes in skin condition or generate or update treatment parameters, in the care routine engine may analyze (e.g., using computer vision and/or machine learning techniques described herein) digital scans to measure or map wrinkles, pigmentation, skin texture, etc., of the user's skin in a single image or a series of images captured over time. In such a scenario, the care routine engine may use such information to recommend, generate or modify a particular care routine that suits the particular features of the user's skin.

In addition to the technical benefits of described embodiments that are described elsewhere herein, numerous other technical benefits are achieved in some embodiments. For example, the system allows some aspects of the process to be conducted independently by personal care devices or client computing devices, while moving other processing burdens to a remote computer system (which may be a relatively high-powered and reliable computing system), thus improving performance and preserving battery life for functionality provided by personal care devices or client computing devices.

In general, the word “engine,” as used herein, refers to logic embodied in hardware or software instructions written in a programming language, such as C, C++, COBOL, JAVA™, PHP, Perl, HTML, CSS, JavaScript, VBScript, ASPX, Microsoft.NET™, and/or the like. An engine may be compiled into executable programs or written in interpreted programming languages. Software engines may be callable from other engines or from themselves. Generally, the engines described herein refer to logical modules that can be merged with other engines or divided into sub-engines. The engines can be stored in any type of computer-readable medium or computer storage device and be stored on and executed by one or more general purpose computers, thus creating a special purpose computer configured to provide the engine or the functionality thereof.

As understood by one of ordinary skill in the art, a “data store” as described herein may be any suitable device configured to store data for access by a computing device. One example of a data store is a highly reliable, high-speed relational database management system (DBMS) executing on one or more computing devices and accessible over a high-speed network. Another example of a data store is a key-value store. However, any other suitable storage technique and/or device capable of quickly and reliably providing the stored data in response to queries may be used, and the computing device may be accessible locally instead of over a network, or may be provided as a cloud-based service. A data store may also include data stored in an organized manner on a computer-readable storage medium, as described further below. One of ordinary skill in the art will recognize that separate data stores described herein may be combined into a single data store, and/or a single data store described herein may be separated into multiple data stores, without departing from the scope of the present disclosure.

The devices shown in FIG. 5 may communicate with each other via a network (not shown), which may include any suitable communication technology including but not limited to wired technologies such as DSL, Ethernet, fiber optic, USB, and Firewire; wireless technologies such as Wi-Fi, WiMAX, 3G, 4G, LTE, 5G, and Bluetooth; and the Internet. In general, communication between computing devices or components in FIG. 5, or other components or computing devices used in accordance with described embodiments, occurs directly or through intermediate components or devices.

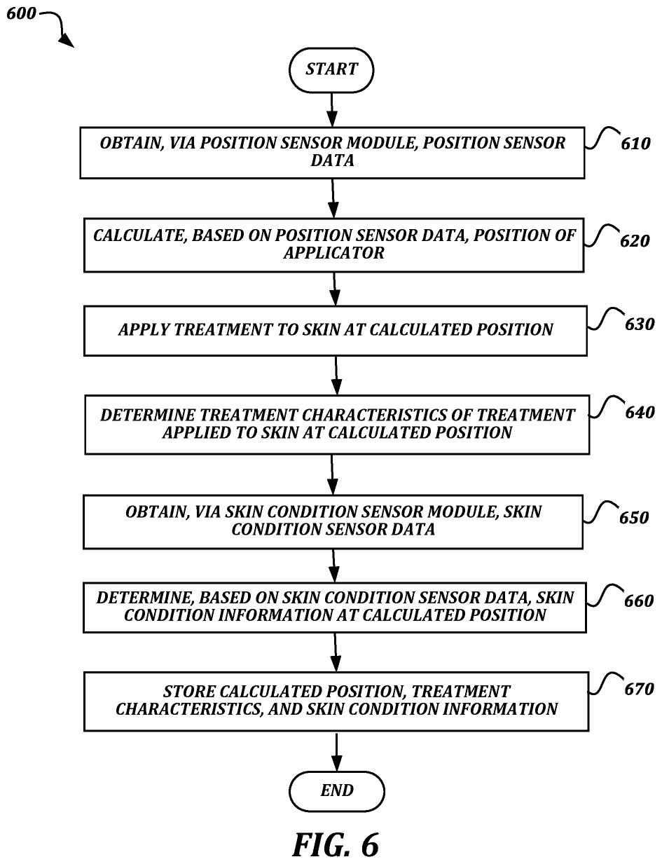

FIG. 6 is an example method 600 of using a personal care system to apply and monitor treatments and determine skin condition, in accordance with the present technology. In some embodiments, the system is system 1000 or system 2000 as shown and described in FIGS. 4 and 5. In some embodiments, the system includes an applicator (such as applicator 100 in FIGS. 1A-5), sensor modules including one or more sensors (as shown in FIGS. 1B, 1C, 2B, 2C, 3A, 3B, and 4), and one or more processors (such as applicator processor 140, 240 or base device processor 255 as described herein). Although the description of FIG. 6 and other descriptions herein refer to “a processor” for ease of explanation, it should be understood that in some embodiments, multiple processors on the same device or on multiple devices may be used to perform the described functions. In some embodiments, the system includes one or more light sources (such as light sources 220 described herein) or one or more radiating elements (such as radiating elements 135 as described herein). In some embodiments, the system further includes a communication device (such as communication device 300) and/or a base device (such as base device 200).

In block 610, the system obtains, via a position sensor module, position sensor data. In some embodiments, the position sensor module includes a camera, a proximity sensor, a gyroscope, an accelerometer, or a combination thereof. In some embodiments, the position sensor data includes still images or video, proximity data, orientation data, acceleration data, or other data useful for calculating position or orientation of an applicator in use.

In block 620, the system calculates, based on the position sensor data, a position of the applicator in use. In some embodiments, the calculated position comprises spatial coordinates, such as 2D coordinates (X/Y) in a 2D space or 3D coordinates (X/Y/Z or mesh coordinates) in a 3D space. In some embodiments, the calculated position of the applicator corresponds to a particular location on the user's skin.

In block 630, the system applies a treatment to the skin at the calculated position, using one or more of the techniques or treatment devices described herein.

In block 640, the system determines treatment characteristics of the treatment applied to the skin at the calculated position. By calculating the position and cross-referencing the treatment characteristics applied at that position, the system is able to store detailed information that allows treatments and the progress of those treatments to be tracked over time.

In some embodiments, the treatment characteristics include a timestamp. In some embodiments, such as for application of a formula to the skin, the treatment characteristics further include an identifier of a dispensed formula, a quantity of the dispensed formula, or a combination thereof. In some embodiments, such as for light treatments, phototherapy treatments, or radiation treatments, the treatment characteristics further include an intensity of treatment, a wavelength of treatment, a duration of treatment, or a combination thereof.

In block 650, the system obtains, via a skin condition sensor module, skin condition sensor data. In some embodiments, the skin condition sensor module includes a camera, a skin hydration sensor, a skin elasticity sensor, or a combination thereof. In some embodiments, the skin condition sensor data includes still images or video, skin hydration data (e.g., capacitance data or conductance data), skin elasticity data (e.g., mechanical resistance data or tonometry data) or a combination thereof.

In block 660, the system determines, based on the skin condition sensor data, skin condition information at the calculated position. In some embodiments, the skin condition sensor data may allow the applicator, the base device, the communication device, or some external computing device (such as a server computer in a cloud computing environment) to determine a condition of a portion of the skin. In some embodiments, the condition is hyperpigmentation, acne, fine lines or wrinkles, sagging, sun damage, hydration level, elasticity, oiliness, or a combination thereof. In some embodiments, determination of skin condition is performed by one or more processors, which may be located on the applicator, the base device, the communication device, or some other computing device (such as a server in a cloud computing environment) or a combination thereof.

In block 670, the system stores the calculated position, treatment characteristics, and skin condition information. Such information may be stored in volatile or non-volatile storage media in the applicator, the base device, the communication device, or some external computing device (such as a server computer in a cloud computing environment).

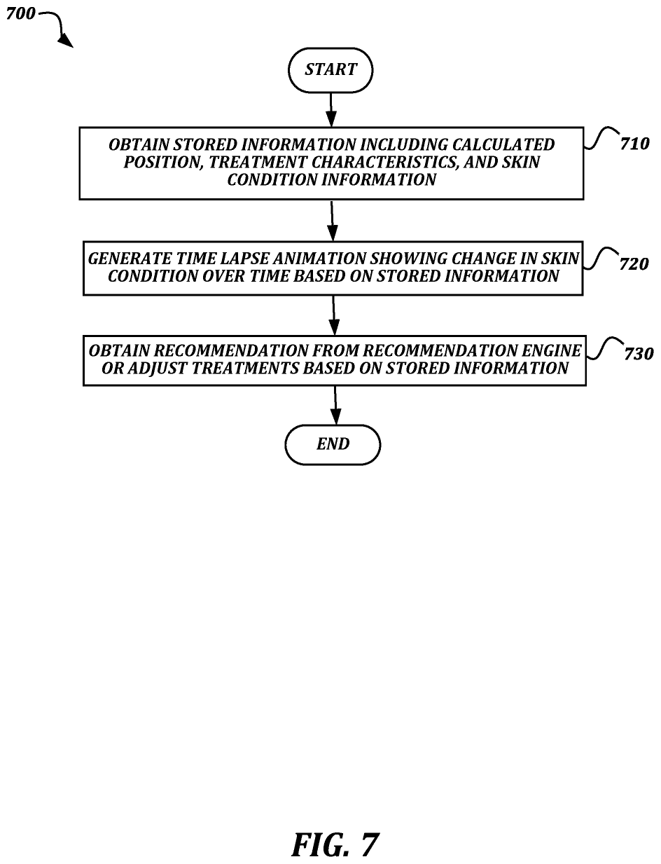

FIG. 7 is an example method 700 of using a personal care system to monitor progress of treatments and make adjustments based on progress of the treatments, in accordance with the present technology. In some embodiments, the system is system 1000 or system 2000 as shown and described in FIGS. 4 and 5. In some embodiments, the system includes an applicator (such as applicator 100 in FIGS. 1A-5), sensor modules including one or more sensors (as shown in FIGS. 1B, 1C, 2B, 2C, 3A, 3B, and 4), and one or more processors (such as applicator processor 140, 240 or base device processor 255 as described herein).

In block 710, the system obtains stored information including treatment characteristics and skin condition information cross-referenced with the calculated position of an applicator that applies the treatment(s) at that position.

In block 720, the system generates a visualization, such as a time lapse animation, of changes in skin condition based on the stored information. This can help show users the progress of skin treatments and suggest ways that treatments may be adjusted to achieve better results. Optionally, the skin condition information is compared with a baseline condition of the skin, such as a past condition of the skin, and an average condition over time, or a desired skin condition. In some embodiments, the baseline condition is stored and/or communicated to the user with the base device or the communication device. In some embodiments, the communication device stores each time the user applies one or more treatments, creating a user history of position information, treatment characteristics, and skin condition information. Optionally, baseline conditions are updated over time based on such information. The visualization may include an image or 3D model of a skin area being treated, such as a 3D mesh object with skin condition features, treatment characteristics, or other information mapped onto the object. In some embodiments, one or more portions of the skin are highlighted. In some embodiments, the one or more portions are highlighted on the communication device, either graphically, or overlaid on a model or an image taken with the one or more sensor modules. In some embodiments, the one or more portions are areas where a treatment has been or not applied, a condition has been detected, a change in condition has been detected, or a combination thereof. In some embodiments, different colors, symbols, patterns, or the like may be used to distinguish areas of interest, such as highlighting areas where treatment has been successful in green, and areas where more treatment is needed in red. An animation of progress of the treatment overtime may include gradual changes in color (e.g., from red to green to show areas responding well to treatment) or other visual characteristics in corresponding skin areas.

In block 730, the system obtains a recommendation from a recommendation engine or adjusts treatments based on the stored information. In some embodiments, the system determines whether to adjust the one or more treatments periodically. In some embodiments, the system adjusts one or more parameters of the one or more treatments (e.g., formula flow rate, formula type, wavelength, intensity, duration, etc.) after a predetermined time interval. In some embodiments, the base device adjusts the one or more treatments after a predetermined time interval. In some embodiments, the system determines whether to adjust one or more parameters of the one or more treatments after each use. In some embodiments the system identifies a product (such as a new or previously used formula) based on the stored information and automatically places an order to obtain the product.

It should be understood that methods 600 and 700 should be interpreted as merely representative. In some embodiments, process blocks of methods 600 and 700 may be performed simultaneously, sequentially, in a different order, or even omitted, without departing from the scope of this disclosure.

Embodiments disclosed herein may utilize circuitry in order to implement technologies and methodologies described herein, operatively connect two or more components, generate information, determine operation conditions, control an appliance, device, or method, and/or the like. Circuitry of any type can be used. In an embodiment, circuitry includes, among other things, one or more computing devices such as a processor (e.g., a microprocessor), a central processing unit (CPU), a digital signal processor (DSP), an application-specific integrated circuit (ASIC), a field-programmable gate array (FPGA), or the like, or any combinations thereof, and can include discrete digital or analog circuit elements or electronics, or combinations thereof.

An embodiment includes one or more data stores that, for example, store instructions or data. Non-limiting examples of one or more data stores include volatile memory (e.g., Random Access memory (RAM), Dynamic Random Access memory (DRAM), or the like), non-volatile memory (e.g., Read-Only memory (ROM), Electrically Erasable Programmable Read-Only memory (EEPROM), Compact Disc Read-Only memory (CD-ROM), or the like), persistent memory, or the like. Further non-limiting examples of one or more data stores include Erasable Programmable Read-Only memory (EPROM), flash memory, or the like. The one or more data stores can be connected to, for example, one or more computing devices by one or more instructions, data, or power buses.

In an embodiment, circuitry includes a computer-readable media drive or memory slot configured to accept signal-bearing medium (e.g., computer-readable memory media, computer-readable recording media, or the like). In an embodiment, a program for causing a system to execute any of the disclosed methods can be stored on, for example, a computer-readable recording medium (CRMM), a signal-bearing medium, or the like. Non-limiting examples of signal-bearing media include a recordable type medium such as any form of flash memory, magnetic tape, floppy disk, a hard disk drive, a Compact Disc (CD), a Digital Video Disk (DVD), Blu-Ray Disc, a digital tape, a computer memory, or the like, as well as transmission type medium such as a digital and/or an analog communication medium (e.g., a fiber optic cable, a waveguide, a wired communications link, a wireless communication link (e.g., transmitter, receiver, transceiver, transmission logic, reception logic, etc.). Further non-limiting examples of signal-bearing media include, but are not limited to, DVD-ROM, DVD-RAM, DVD+RW, DVD-RW, DVD-R, DVD+R, CD-ROM, Super Audio CD, CD-R, CD+R, CD+RW, CD-RW, Video Compact Discs, Super Video Discs, flash memory, magnetic tape, magneto-optic disk, MINIDISC, non-volatile memory card, EEPROM, optical disk, optical storage, RAM, ROM, system memory, web server, or the like.

The detailed description set forth above in connection with the appended drawings, where like numerals reference like elements, are intended as a description of various embodiments of the present disclosure and are not intended to represent the only embodiments. Each embodiment described in this disclosure is provided merely as an example or illustration and should not be construed as preferred or advantageous over other embodiments. The illustrative examples provided herein are not intended to be exhaustive or to limit the disclosure to the precise forms disclosed. Similarly, any steps described herein may be interchangeable with other steps, or combinations of steps, in order to achieve the same or substantially similar result. Generally, the embodiments disclosed herein are non-limiting, and the inventors contemplate that other embodiments within the scope of this disclosure may include structures and functionalities from more than one specific embodiment shown in the figures and described in the specification.

The principles, representative embodiments, and modes of operation of the present disclosure have been described in the foregoing description. However, aspects of the present disclosure, which are intended to be protected, are not to be construed as limited to the particular embodiments disclosed. Further, the embodiments described herein are to be regarded as illustrative rather than restrictive. It will be appreciated that variations and changes may be made by others, and equivalents employed, without departing from the spirit of the present disclosure. Accordingly, it is expressly intended that all such variations, changes, and equivalents fall within the scope of the present disclosure as claimed.

The embodiments of the invention in which an exclusive property or privilege is claimed are defined as follows:

1. A personal care system comprising:
an applicator including a body and an applicator surface coupled to the body, wherein the applicator surface is configured to apply a formula to skin;
a position sensor module;
a skin condition sensor module;
a processor; and
memory having stored therein computer-executable instructions configured to cause the personal care system to perform steps comprising:
obtaining, via the position sensor module, position sensor data;
calculating, based on the position sensor data, a position of the applicator on the skin;
applying a treatment to the skin at the calculated position;
determining treatment characteristics of the treatment applied to the skin at the calculated position;
obtaining, via the skin condition sensor module, skin condition sensor data;
determining, based on the skin condition sensor data, skin condition information at the calculated position; and
storing the calculated position, the treatment characteristics, and the skin condition information.

2. The personal care system of claim 1 wherein the skin condition sensor module includes a skin hydration sensor, a skin elasticity sensor, or a combination thereof.

3. The personal care system of claim 1 wherein the position sensor module includes a camera, a proximity sensor, a gyroscope, an accelerometer, or a combination thereof.

4. The personal care system of claim 1, wherein the treatment characteristics include a timestamp.

5. The personal care system of claim 4, wherein the treatment characteristics further include an identifier of a dispensed formula, a quantity of the dispensed formula, or a combination thereof.

6. The personal care system of claim 4, wherein the treatment characteristics further include an intensity of treatment, a wavelength of treatment, a duration of treatment, or a combination thereof.

7. The personal care system of claim 1, wherein the skin condition information comprises an image of the skin at the calculated location, skin elasticity information, skin hydration information, or a combination thereof.

8. The personal care system of claim 1, wherein the computer-executable instructions are further configured to cause the personal care system to compare the determined skin condition information with a baseline condition and adjust one or more treatments based on the comparison.

9. The personal care system of claim 1 further comprising one or more light sources, and wherein treatment comprises a light treatment or phototherapy treatment.

10. A method performed by a computer system comprising one or more processors and memory, the method comprising, by the one or more processors:
obtaining position sensor data;
calculating, based on the position sensor data, a position of an applicator relative to skin of a user;
determining treatment characteristics of a treatment applied to the skin at the calculated position;
obtaining skin condition sensor data;
determining, based on the skin condition sensor data, skin condition information of the skin at the calculated position; and
storing the calculated position, the treatment characteristics, and the skin condition information.

11. The method of claim 10, wherein the skin condition information comprises an image of the skin at the calculated location, skin elasticity information, skin hydration information, or a combination thereof.

12. The method of claim 10, wherein the treatment characteristics include a timestamp.

13. The method of claim 12, wherein the treatment characteristics further include an identifier of a dispensed formula, a quantity of the dispensed formula, or a combination thereof.

14. The method of claim 12, wherein the treatment characteristics further include an intensity of treatment, a wavelength of treatment, a duration of treatment, or a combination thereof.

15. The method of claim 10 further comprising comparing the determined skin condition information with a baseline condition and adjusting one or more treatments based on the comparison.

16. The method of claim 10 further comprising generating a visualization of a change in skin condition over time based on the stored calculated position, the stored treatment characteristics, and the stored skin condition information.

17. The method of claim 10 further comprising obtaining a recommendation from a recommendation engine or adjusting one or more treatments based on analysis of the stored information.

18. A non-transitory computer-readable medium having stored thereon computer-executable instructions configured to cause one or more processors of a computer system to perform steps comprising:
obtaining position sensor data;
calculating, based on the position sensor data, a position of an applicator relative to skin of a user,
determining treatment characteristics of a treatment applied to the skin at the calculated position;
obtaining skin condition sensor data;
determining, based on the skin condition sensor data, skin condition information of the skin at the calculated position; and
storing the calculated position, the treatment characteristics, and the skin condition information.

19. The non-transitory computer-readable medium of claim 18, wherein the stored skin condition information comprises an image of the skin at the calculated location, skin elasticity information, skin hydration information, or a combination thereof, and wherein the stored treatment characteristics include a timestamp and one or more of an identifier of a dispensed formula, a quantity of the dispensed formula, an intensity of treatment, a wavelength of treatment, or a duration of treatment.

20. The non-transitory computer-readable medium of claim 18, the steps further comprising generating a visualization of a change in skin condition over time based on the stored calculated position, the stored treatment characteristics, and the stored skin condition information.
